(19) United States
(12) Patent Application Publication
(10) Pub. No.: US 2014/0263548 A1
(43) Pub. Date: Sep. 18, 2014

(54) SURGICAL FASTENER APPLYING APPARATUS

(71) Applicant: Covidien LP, Mansfield, MA (US)

(72) Inventors: Mark Tanner, LONDON (GB); Robbie Wilson, HARWICH (GB)

(73) Assignee: Covidien LP, Mansfield, MA (US)

(21) Appl. No.: 14/197,773

(22) Filed: Mar. 5, 2014

Related U.S. Application Data
(60) Provisional application No. 61/791,818, filed on Mar. 15, 2013.

(51) Int. Cl. A61B 17/068

(52) U.S. Cl. CPC A61B 17/068
USPC 227/175.2; 227/175.1

(57) ABSTRACT

A reusable surgical stapling device is provided. The stapling device includes a staple pusher assembly and an approximation assembly. The stapling device further includes a handle assembly including first and second handle sections pivotally secured in relation to each other about a proximal portion of the staple pusher and approximation assemblies. The first and second handle sections being pivotal between an open configuration and a closed configuration. When in the open configuration, the first and second handle sections are pivoted to expose the proximal portion of the staple pusher and approximation assemblies. An outer tube is selectively received about a distal portion of the staple pusher and approximation assemblies. A ferrule is received about a proximal end of the outer tube and is releasably connected to a distal end of the first and second handle sections to secure the outer tube to the handle assembly and retain the first and second handle sections in a closed configuration.

### CROSS-REFERENCE TO RELATED APPLICATIONS

This application claims the benefit of and priority to U.S. Provisional Patent Application No. 61/791,818, filed Mar. 15, 2013, the entire disclosure of which is incorporated by reference herein.

### BACKGROUND

1. Technical Field

The present disclosure relates to a surgical fastener applying apparatus and, more particularly, to surgical fastener applying apparatus having reusable and disposable components.

2. Discussion of Related Art

Anastomosis refers to the surgical joining of separate hollow tissue sections. Typically, an anastomosis procedure follows surgery in which a diseased or defective section of a hollow tissue structure is removed, thus requiring the joining of the remaining end sections of the tissue structure. Depending on the particular procedure being performed and/or other factors, the end sections of the tissue may be joined by circular anastomosis, e.g., end-to-end anastomosis, end-to-side anastomosis, or side-to-side anastomosis.

In a circular anastomosis procedure, the two end sections of a tubular organ are joined using a stapling apparatus that drives a circular array of staples through each of the end sections to join the end sections to one another in end-to-end relation and simultaneously cores any tissue within the newly joined hollow tissue structure to clear the passage defined by the hollow tissue structure. The apparatus can also apply other surgical fasteners such as, for example, clips or two part polymeric surgical fasteners.

A typical circular anastomosis apparatus includes an elongated shaft having a handle portion at a proximal end and a fastener holding component at a distal end. An anvil assembly including an anvil rod and an attached anvil head is mounted to the distal end of the elongated shaft adjacent the fastener holding component. In use, the end portions to be joined are clamped between the anvil head and the fastener holding component. The clamped end portions are then joined to one another by driving one or more staples from the fastener holding component, through the tissue, and into the anvil head to form the fasteners about the tissue. In some such apparatus, a knife is provided to cut the tissue which has been joined by the staples. An example of such a circular anastomosis apparatus is described in U.S. Pat. Nos. 7,857,187 and 7,303,106 to Milliman, the entire content of each of which is hereby incorporated by reference herein in their entirety.

Typically, the entire surgical stapling apparatus for performing a circular anastomosis procedure is disposable after a single use. A need exists for a surgical stapling apparatus that includes reusable components and is not overly complex and is configured to facilitate sterilization after use in a surgical procedure.

### SUMMARY

A surgical stapling device is provided which includes a staple pusher assembly, an approximation assembly, a handle assembly including first and second handle sections releasably secured about a proximal portion of the staple pusher and approximation assemblies, an outer tube selectively received about a distal portion of the staple pusher and approximation assemblies, and a ferrule configured to be received about a proximal end of the outer tube and releasably secured to a distal end of the first and second handle sections for securing the outer tube to the handle assembly and maintaining the first and second handle sections in the closed configuration. The first and second handle sections are movable between an open configuration and a closed configuration, wherein in the open configuration, the first and second handle sections expose the staple pusher and approximation assemblies.

In an embodiment, the stapling device further includes a knob releasably attached to a distal end of the approximation assembly. The stapling device may further include a base, wherein the first and second handle sections are pivotally secured to the base. A longitudinal edge of each of the first and second handle sections may be configured for pivotal engagement with the base. A proximal end of each of the first and second handle sections may be configured for pivotal engagement with the base. A proximal extension of the base may include a pair of semi-circular flanges configured to be selectively received about a rotatable sleeve of the approximation assembly. A distal extension of the base may include an semi-circular flange configured to be releasably received about an elongated pusher tube of the staple pusher assembly.

In some embodiments, the base includes a pair of support members extending therefrom for stabilizing a firing trigger of the staple pusher assembly. The ferrule may engage the distal end of the first and second handle sections by friction-fit. The ferrule may engage the distal end of the first and second handle sections by bayonet coupling. Alternatively, the ferrule may engage the distal end of the first and second handle sections by threads.

Also disclosed is a method of assembling a surgical stapler. The method includes a step of providing a staple pusher assembly, an approximation assembly, a housing assembly, an outer tube and a ferrule, wherein the housing assembly includes first and second handle sections. The method further includes the steps of receiving the outer tube and the ferrule over a distal end of the staple pusher and a distal end of the approximation assembly, attaching the handle assembly to a proximal portion of approximation assembly, positioning a proximal end of the outer tube between distal end of the first and second handle sections, pivoting first and second handle sections of the handle assembly relative to each other to a closed position to encase the proximal portions of staple pusher and approximation assemblies, and securing the ferrule to the distal ends of the first and second handle sections.

In addition, a method of disassembling a surgical stapler is provided. The method includes a step of providing a surgical stapler having a staple pusher assembly, an approximation assembly, a housing assembly, an outer tube and a ferrule, wherein the housing assembly includes first and second handle sections. The method further includes the steps of disengaging the ferrule from about distal ends of the first and second handle sections, pivoting at least one of the first and second handle sections relative to each other to expose proximal ends of the staple pusher assembly and the approximation assembly, removing the outer tube and ferrule from about distal ends of the staple pusher assembly and the approximation assembly, and disengaging the housing assembly from the proximal ends of the staple pusher assembly and the approximation assembly.

### BRIEF DESCRIPTION OF THE DRAWINGS

Various embodiments of the presently disclosed surgical fastener applying apparatus will now be described herein with reference to the accompanying figures wherein:

FIG. 1 is a perspective view of a staple pusher assembly and an approximation assembly of a surgical stapling device;

FIG. 2 is a perspective view of a housing assembly according to an embodiment of the present disclosure;

FIG. 3 is a perspective view of the staple pusher assembly and the approximation assembly of FIG. 1 including an outer tube, a ferrule and an approximation knob of the housing assembly of FIG. 2;

FIG. 4 is a perspective view of the staple pusher assembly and the approximation assembly of FIG. 1 including the housing assembly of FIG. 2 received thereabout in an open configuration;

FIG. 5 is a perspective view of the staple pusher assembly and the approximation assembly of FIG. 1 including the housing assembly of FIG. 2 received thereabout in a closed configuration;

FIG. 6 is a perspective view of the staple pusher assembly and the approximation assembly of FIG. 1 including the housing assembly of FIG. 2 received thereabout in a closed and secured configuration;

FIG. 7 is a perspective view of the staple pusher assembly and the approximation assembly of FIG. 1 including a housing assembly according to an alternate embodiment of the present disclosure received thereabout in a closed and secured configuration;

FIG. 8 is a perspective view of the staple pusher assembly and the approximation assembly of FIG. 1 including a first handle section, an outer tube, a ferrule and an approximation knob of the housing assembly of FIG. 7; and

FIG. 9 is a perspective view of the staple pusher assembly and the approximation assembly of FIG. 1 including the first handle section, the outer tube, the ferrule and the approximation knob of FIG. 8 with the first handle section secured to the staple pusher and approximation assemblies;

FIG. 10 is a perspective view of the staple pusher assembly and the approximation assembly of FIG. 1 and the housing assembly of FIG. 7 received thereabout in an open configuration;

FIG. 11 is a perspective view of the staple pusher assembly and the approximation assembly of FIG. 1 and the housing assembly of FIG. 7 received thereabout in a closed configuration;

FIG. 12 is a perspective view of a housing assembly according to another embodiment of the present disclosure;

FIG. 13 is a perspective view of the staple pusher assembly and the approximation assembly of FIG. 1 including an outer tube and ferrule of the housing assembly of FIG. 12;

FIG. 14 is a perspective view of the staple pusher assembly and the approximation assembly of FIG. 1 and the housing assembly of FIG. 12 in an open configuration;

FIG. 15 is a perspective view of the staple pusher assembly and the approximation assembly of FIG. 1 and the housing assembly of FIG. 12 in a closed configuration; and

FIG. 16 is a perspective view of the staple pusher assembly and the approximation assembly of FIG. 1 and the housing assembly of FIG. 12 in a closed and secured configuration.

### DETAILED DESCRIPTION OF EMBODIMENTS

Embodiments of the presently disclosed surgical stapling device will now be described in detail with reference to the drawings in which like reference numerals designate identical or corresponding elements in each of the several views. Throughout this description, the term “proximal” will refer to the portion of the instrument closer to the operator and the term “distal” will refer to the portion of the instrument further from the operator.

Each of the presently disclosed surgical stapling devices include a housing assembly which is configured to releasably retain the internal components of the surgical stapling device and support a staple assembly of the surgical stapling device. As will become apparent from the following disclosure, the construction of the housing assemblies facilitates the sterilization of the internal components of the stapling device between uses. It is also envisioned that the components of the housing assemblies may also be capable of sterilization.

With particular reference to FIG. 1, the internal components of the surgical stapling device include an approximation assembly 50 and a staple pushing assembly 70. Approximation assembly 50 and staple pushing assembly 70 will only be described to the extent necessary to fully disclose the housing assemblies of the present disclosure.

Briefly, and with reference to FIG. 1, staple pusher assembly 50 includes a firing trigger 52 and a pusher linkage 60. Firing trigger 52 includes a firing link 54. As shown, firing link 54 is integrally formed with firing trigger 52. Firing link 54 and firing trigger 52 are pivotal relative to each other through operation of a living hinge 53. Alternatively, firing trigger 52 and firing link 54 may be formed as separate components that are pivotally connected to each by pivot pin or by any other suitable means. Pusher linkage 60 includes an elongated pusher tube 62, a pusher link 64 and a pusher end tube 66. A proximal end 62a of elongated pusher tube 62 is operably connected to firing trigger 52. Proximal end 62a of elongated pusher tube 62 includes a pair of extensions 63. As will be discussed in further detail below, extensions 63 are configured to be slidably received within a slot (115, FIG. 2) formed in the handle assembly of the housing assembly to constrain elongated pusher tube 62 to linear movement within handle assembly 110. A distal end 62b of elongated pusher tube 64 is pivotally connected to a proximal end 64a of pusher link 64 and a distal end 64b of pusher link 64 is pivotally connected to a proximal end 66a of pusher end tube 66. The pivotal connections facilitate movement of pusher linkage 60 through an outer tube 140 of a housing assembly 100 (FIG. 2). A distal end 66a of pusher end tube 66 is configured for operable connection with a pusher back (not shown) of a staple assembly (not shown).

With reference still to FIG. 1, approximation assembly 70 includes a rotatable sleeve 72, a screw 74, a screw extension 76, and an anvil retainer 78. A proximal end 72a of rotatable sleeve 72 is configured for selective engagement with an approximation knob. A distal end 72b of rotatable sleeve 72 includes a collar 73. Screw 74 is selectively movable within rotatable sleeve 72 in response to rotation of sleeve 72 about screw 74. A proximal end (not shown) of screw extension 76 is secured to a distal end (not shown) of screw 74 and a distal end 76b of screw extension 76 is secured to a proximal end 78a of an anvil retainer 78. Screw 74, screw extension 76 and anvil retainer 78 are slidably disposed through pusher linkage 60 of staple pusher assembly 50 to control movement of an anvil which may be secured to anvil retainer 78.

With reference now to FIGS. 2-6, an embodiment of a housing assembly for supporting staple pusher assembly 50 and approximation assembly 70 is shown generally as housing assembly 100. Housing assembly 100 includes a handle assembly 110, an outer tube 140 configured to extend distally from handle assembly 110 and, a ferrule 145 for maintaining handle assembly 110 in a closed position (FIG. 5). An approximation knob 150 is configured for selective engagement with proximal end 72a of rotatable sleeve 72 of approximation assembly 70.

With particular reference now to FIGS. 2 and 3, handle assembly 110 includes first and second handle sections 112a, 112b and a chassis or frame 130. First and second handle sections 112a, 112b are each pivotally connected to chassis 130 along a first longitudinal edge 114a, 114b, respectively. In certain embodiments, first and second handle sections 112a, 112b may be connected to chassis 130 using pins (not shown), one or more hinges (not shown) or any other suitable means that permits pivoting of first and second handle sections 112a, 112b relative to each other and to chassis 130 between an open configuration (FIG. 4) and a closed configuration (FIG. 5). In the open configuration of first and second handle sections 112a, 112b, the proximal components of approximation assembly 70 and pusher assembly 50 can be accessed and/or separated from the handle assembly 110 to facilitate sterilization of these components. Alternatively, first and second handle sections 112a, 112b may be integrally formed with chassis 130 and connected thereto by a living hinge.

Second longitudinal edges 116a, 116b of first and second handle sections 112a, 112b, respectively, each define a cutout 113a, 113b. When first and second handle sections 112a, 112b are in a closed configuration (FIG. 5), cutouts 113a, 113b collectively form an opening 113 through which firing trigger 52 of staple pusher assembly 50 extends. Although not shown, second longitudinal edges 116a, 116b of first and second handle sections 112a, 112b, respectively, may include a latch mechanism (not shown) or other means for selectively maintaining first and second handle sections 112a, 112b in the closed configuration. The latch mechanism may be, for example, any snap-fit type connector or releasable interlocking member formed adjacent respective longitudinal edges 116a, 116b.

With continued reference to FIGS. 2 and 3, handle sections 112a, 112b each define a slot 115 configured to receive extension 63 formed in proximal end 62a of elongated pusher tube 62 of staple pusher assembly 50. As discussed above, slots 115 constrain elongated pusher tube 62 to linear movement within handle assembly 110 during firing of the surgical stapling device. Handle sections 112a, 112b each include a flange 118 (FIG. 4) which is configured to engage collar 73 formed on distal end 72b of rotatable sleeve 72 of approximation assembly 70 to axially fix collar 73 within handle assembly 110. Distal ends 120a, 120b of first and second handle sections 112a, 112b, respectively, together define a threaded collar 120 (FIG. 5) configured for selective engagement by ferrule 145 when first and second handle sections 112a, 112b are in the closed configuration. Alternatively, the distal end of handle assembly 110 may selectively receives ferrule 145 in a manner commonly referred to as a bayonet coupling. In this manner, one of the distal end of handle assembly 110 and ferrule 145 includes one or more tabs (not shown) extending radially outward therefrom configured to be selectively received within one or more corresponding L-shaped slots (not shown) formed in the other of the distal end of handle assembly 110 and ferrule 145. Once handle assembly 110 and ferrule 145 are positioned such that the tabs are received within the a transverse portion of the slots, handle assembly 110 and ferrule 145 can be rotated relative to each other along their longitudinal axes to selectively secure ferrule 145 to handle assembly 110. In another embodiment, the distal end of handle assembly 110 is configured to selectively receive ferrule 145 in a friction-fit manner.

The distal end of handle assembly 110 is further configured to receive the proximal end 140a of outer tube 140. In an embodiment, the distal end of handle assembly 110 defines an annular groove (not shown) which receives a flange or annular rib (not shown) formed on a proximal end 140a of outer tube 140 to releasably secure outer tube 140 with the distal end of handle assembly 110. Alternatively, other interlocking configurations are envisioned. As will be discussed in further detail below, during assembly of housing assembly 100, proximal end 140a of outer tube 140 is received between distal ends 120a, 120b of first and second handle sections 112a, 112b, respectively, as first and second handle sections 112a, 112b are pivoted into the closed configuration. Closure of first and second handle sections 112a, 112b about proximal end 140a of outer tube 140 secures outer tube 140 to handle assembly 110.

Referring to FIGS. 2 and 4, chassis 130 of handle assembly 110 includes a base 132 which extends substantially the length of handle sections 112a, 112b. A proximal portion 134 of base 132 includes a pair of semi-circular clips 134a, 134b. Clips 134a, 134b are configured to releasably engage rotatable sleeve 72 of approximation assembly 70. In particular, clips 134a, 134b are configured to selectively receive rotatable sleeve 72 in a snap-fit manner and are sized to permit rotation of rotatable sleeve 72 along its longitudinal axis. A distal portion 136 of base 132 includes a semi-circular flange or clip 136a configured to releasably engage elongated pusher tube 62. In particular, clip 136a is configured to releasably receive elongated pusher tube 62 in a snap-fit manner and is sized to permit longitudinal translation of elongated pusher tube 62 therethrough. A pair of supports 138a, 138b extends from base 132 between proximal portion 134 and distal portion 132. Supports 138a, 138b are positioned on base 132 such that an end 54a (FIG. 3) of firing link 54 extending from firing trigger 52 of staple pusher assembly 50 engages supports 138a, 138b when base 132 is attached to elongated pusher tube 62 of staple pusher assembly 50 and rotatable sleeve 72 of approximation assembly 70. During operation of staple pusher assembly 50, supports 138a, 138b maintain end 54a of firing link 54 in axially fixed position.

With continued reference to FIGS. 2 and 3, a distal end 140b of outer tube 140 is configured to releasably engage a shell (not shown) of a staple assembly (not shown). Distal end 140b of outer tube 140 may include tabs (not shown), threads (not shown), slots (not shown) or be otherwise configured to facilitate releasable engagement of outer tube 140 with the shell of the staple assembly.

Still referring to FIGS. 2 and 3, as discussed above, approximation knob 145 is configured to be releasably received on proximal end 72a of rotatable sleeve 72 of approximation assembly 70. Approximation knob 145 may be selectively secured to proximal end 72a of rotatable sleeve 72 through a friction-fit connection, threading, snap-fit or interlocking connectors or by other suitable means. Approximation knob 145 is configured for operable engagement by a user to facilitate rotation of rotable sleeve 72 and may be disposable or, instead, may be configured for sterilization and reuse.

The attachment of housing assembly 100 to staple pusher assembly 50 and approximation assembly 70 will now be described with reference to FIGS. 3-6. Referring initially to FIG. 3, a distal end of approximation assembly 70, including screw extension 76 (FIG. 1) and anvil retainer 78, is received through pusher linkage 60 of staple pusher assembly 50. Approximation knob 150 is then attached to proximal end 72a of rotatable sleeve 72 of approximation assembly 70 and outer tube 140 is slid over pusher linkage 60 of staple pusher assembly 50. Alternatively, approximation knob 150 may be attached to proximal end 72a of rotatable sleeve 72 of approximation assembly 70 subsequent to reception of the distal end of approximation assembly 70 through pusher linkage 60 of staple pusher assembly 50. It is envisioned that approximation knob 150 may be attached to rotatable sleeve 72 at any time during the attachment of housing assembly 100 to staple pusher assembly 50 and approximation assembly 70. Outer tube 140 may also be slid over pusher linkage 60 prior to or subsequent to reception of the distal end of approximation assembly 70 through pusher linkage 60 of staple pusher assembly 50. In addition, ferrule 145 may be slidably positioned over outer tube 140 prior to or subsequent to reception of the distal end of staple pusher assembly 50 through outer tube 140.

Turning now to FIG. 4, handle assembly 110 is releasably attached to staple pusher assembly 50 and approximation assembly 70. In particular, clips 134a, 134b which extend from proximal portion 134 of base 132 of chassis 130 are snap-fit about rotatable sleeve 72 of approximation assembly 70 and clip 136a, which extends from distal portion 136 of base 132, is snap-fit about elongated pusher tube 62 of staple pusher assembly 50. The end 54a of firing link 54 which extends from firing trigger 52 of staple pusher assembly 50 is positioned such that firing link 54 engages the ends of supports 138a, 138b which extend from base 132 of chassis 130.

With reference now to FIG. 5, once base 132 is attached to staple pusher assembly 50 and approximation assembly 70 and firing link 54 is engaged with supports 138a, 138b, first and second handle sections 112a, 112b are pivoted to the closed configuration about the proximal portions of staple pusher assembly 50 and approximation assembly 70. Prior to pivoting handle sections 112a, 112b to the closed configuration, proximal end 140a of outer tube 140 is disposed between distal ends 120a, 120b of first and second handle sections 112a, 112b, respectively, such that outer tube 140 is secured within proximal end 120 of handle assembly 110. As noted above, in the closed configuration, cutouts 113a, 113b formed along second longitudinal edges 116a, 116b, respectively, of respective first and second handle sections 112a, 112b form opening 113 which receives firing trigger 52 of staple pusher assembly 50.

With reference also to FIG. 6, once first and second handle sections 112a, 112b are in the closed configuration, ferrule 145 is moved into engagement with the threaded collar 120 of handle assembly 110, as indicated by arrow “A” in FIG. 5, to maintain first and second handle sections 112a, 112b in the closed configuration. A staple assembly (not shown) may then be attached to distal end 66b of pusher end tube 66 (FIG. 1) and distal end 140b of outer tube 140. The surgical stapling device including housing assembly 100 may then be used in a traditional manner.

After use of the surgical stapling device, the components of the surgical stapling device can be disassembled for sterilization by reversing the steps outlined above. Specifically, once the staple assembly (not shown) is disconnected from outer tube 140 and staple pusher assembly 50, ferrule 145 can be disconnected from distal end 120 of handle assembly 110 and the first and second handle sections 112a, 112b can be pivoted to the open configuration. In the open configuration, outer tube 140 may be removed from about the distal end of staple pusher assembly 50 and from about the distal end of approximation assembly 70. Alternatively, base 132 of chassis 130 may be disconnected from elongated pusher tube 62 of staple pusher assembly 50 and rotatable sleeve 72 of approximation assembly 70. First and second handle sections 112a, 112b may remain attached to base 132 of chassis 130, or, as discussed above, first and second handle sections 112a, 112b may be separated from base 132 to facilitate sterilization of handle assembly 110 and/or to permit replacement of a damaged or worn component. At any point during the disassembly of housing assembly 100, approximation knob 150 may be disengaged from proximal end 72a of rotatable sleeve 70. Once housing assembly 100 is separated from staple pusher assembly 50 and approximation assembly 70, staple pusher assembly 50 and approximation assembly 70 may be separated from each other and sterilized. As discussed above, any or all of the components of housing assembly 100 may be sterilized and/or replaced prior to reuse.

Turning now to FIGS. 7-11, an alternative embodiment of a housing assembly according to the present disclosure is shown generally as housing assembly 200. Housing assembly 200 is configured for supporting staple pusher assembly 50 and approximation assembly 70. Housing assembly 200 includes a handle assembly 210, an outer tube 240 which extends distally from handle assembly 210, a ferrule 245 for maintaining handle assembly 210 in a closed position (FIG. 7) secured to the proximal end of outer tube 240, and an approximation knob 250 adapted to be releasably engaged with proximal end 72a of rotatable sleeve 72. As will become apparent from the following description, handle assembly 210 is configured for releasable engagement with a proximal portion of staple pusher assembly 50 and a proximal portion of approximation assembly 70. Further, the outer tube 240 is configured to releasably receive a distal portion of the staple pusher assembly 50 and a distal portion of the approximation assembly 70.

With continued reference to FIGS. 7-11, handle assembly 210 includes a first handle section 212 and a second handle section 222 (FIG. 10). First and second handle sections 212, 222 are releasably connected to one another at proximal ends 212a, 222a, respectively. As shown, first handle section 212 defines a pair of notches 214 on proximal end 212a thereof configured to receive a pair of flanges 224 formed on proximal end 222a of second handle section 222. Although shown with notches 214 defined on first handle section 212 and flanges 224 formed on second handle section 222, it is envisioned that second handle section 222 may instead define the notches and first handle section 212 may include the flanges. It is further envisioned that proximal ends 212a, 222a of first and second handle sections 212, 222, respectively, may be selectively connected using any suitable means, including, for example, a pin and slot configuration.

With continued reference to FIGS. 7-11, first handle section 212 of handle assembly 210 includes an opening (not shown) through which firing trigger 52 and firing link 54 of staple pusher assembly 50 extend when first handle section 212 is received about the proximal ends of staple pusher and approximation assemblies 50, 70. First handle section 212 further includes a flange 218 (FIG. 8) configured to engage collar 73 formed on distal end 72b of rotatable sleeve 72 of approximation assembly 70. Although not shown, second handle section 222 may also include a flange (not shown) for engaging collar 73 of approximation assembly 70. As discussed above with regard to handle sections 12a, 12b, first handle section 212 may define a pair of slots for receiving extensions 63 formed on proximal end 62a of elongated pusher tube 62 of staple pusher assembly 50 for constraining pusher linkage 60 to linear movement upon actuation of firing trigger 52.

Distal ends 212b, 222b of first and second handle sections 212, 222, respectively, together define a threaded collar 210b configured for releasable engagement with ferrule 245 when first and second handle sections 212, 222 are in the closed configuration. Alternatively, distal end 210b of handle assembly 210 may define a bayonet type coupling adapted to releasably engage ferrule 245, as discussed above. In another embodiment, distal end 210b of handle assembly 210 may be configured to selectively receive ferrule 245 in a friction-fit manner. It is envisioned that ferrule 245 may be connected to handle assembly 210 in other suitable manners, including, for example, through a snap-fit connection (not shown) and releasable mechanical fasteners (not shown).

Still referring to FIGS. 7-11, outer tube 240, ferrule 245 and approximation knob 250 of housing assembly 200 are substantially similar in form and function to outer tube 140, ferrule 145 and approximation knob 150 of housing assembly 100, described above.

The attachment of housing assembly 200 to staple pusher assembly 50 and approximation assembly 70 will now be described with reference to FIGS. 7-11. Referring initially to FIG. 8, after the distal end of approximation assembly 70, including screw extension 76 (FIG. 1) and anvil retainer 78, is operable received through pusher linkage 60 of staple pusher assembly 50, approximation knob 250 is attached to proximal end 72a of rotatable sleeve 72 of approximation assembly 70 and outer tube 240 is slid over pusher linkage 60 of staple pusher assembly 50. Alternatively, approximation knob 250 may be attached to proximal end 72a of rotatable sleeve 72 of approximation assembly 70 subsequent to reception of the distal end of approximation assembly 70 through pusher linkage 60 of staple pusher assembly 50. It is envisioned that approximation knob 250 may be attached to rotatable sleeve 72 at any time during the attachment of housing assembly 100 to staple pusher assembly 50 and approximation assembly 70. Outer tube 240 may also be slid over pusher linkage 60 prior to or subsequent to reception of the distal end of approximation assembly 70 through pusher linkage 60 of staple pusher assembly 50. In addition, ferrule 245 is slidably positioned over outer tube 140 prior to or subsequent to reception of the distal end of staple pusher assembly 50 through outer tube 140.

Turning now to FIG. 9, first handle section 212 of handle assembly 210 is releasably attached to staple pusher assembly 50 and approximation assembly 70. In particular, firing trigger 52 and firing link 54 of staple pusher assembly 50 are received through the opening (not shown) formed in first handle section 212 such that flange 218 formed in first handle section 212 engages collar 73 formed on distal end 72b of rotatable sleeve 72 of approximation assembly 70.

With reference now to FIG. 10, once first handle section 212 of handle assembly 210 is attached to staple pusher assembly 50 and approximation assembly 70, each of flanges 224 formed on proximal end 222b of second handle section 222 are engaged with corresponding notches 214 formed on proximal end 212a of first handle section 212, and second handle section 222 is pivoted into the closed configuration with first handle section 212.

With reference back to FIG. 7, once first and second handle sections 212, 222 are in the closed configuration, ferrule 245 is moved into engagement with threaded collar 210b of handle assembly 210, as indicated by arrow “B” in FIG. 7, to maintain first and second handle sections 212, 222 in the closed configuration. A staple assembly (not shown) may then be attached to distal end 66b of pusher end tube 66 (FIG. 1) and distal end 240b of outer tube 240. The surgical stapling device may then be used in a traditional manner.

After use of the surgical stapling device, the components of the surgical stapling device can be disassembled for sterilization by reversing the steps outlined above. Specifically, once the staple assembly (not shown) is disconnected from outer tube 240 and staple pusher assembly 50, ferrule 245 can be disconnected from distal end 210b of handle assembly 210 and the second handle section 222 is disengaged from first handle section 212 by moving distal end 222b of second handle section 222 away from distal end 212b of first handle section 212 and disengaging flanges 224 of second handle section 222 from notches 214 of first handle section 212. In the open configuration, outer tube 240 may be removed from the distal ends of staple pusher and approximation assemblies 50, 70. Alternatively, first handle section 212 may be removed from about firing trigger 52 and firing link 54 of staple pusher assembly 50 prior to removal of outer tube 240. At any point during the disassembly of housing assembly 200, approximation knob 250 may be disengaged from proximal end 72a of rotatable sleeve 70. Once housing assembly 200 is separated from staple pusher assembly 50 and approximation assembly 70, staple pusher assembly 50 and approximation assembly 70 may be separated from each other and sterilized. As discussed above, any or all of the components of housing assembly 200 may be sterilized or replaced prior to reuse.

Turning now to FIGS. 12-16, another embodiment of a housing assembly according to the present disclosure is shown generally as housing assembly 300. Housing assembly 300 is configured for supporting staple pusher assembly 50 and approximation assembly 70. Housing assembly 300 is substantially similar to housing assembly 300 and will only be described as relates to the differences therebetween. Housing assembly 300 includes a handle assembly 310, an outer tube 340 configured to extend form handle assembly 310 and, a ferrule 345 for maintaining handle assembly 310 in a closed configuration (FIG. 16). As will become apparent from the following description, handle assembly 310 is configured for releasable engagement with proximal portions of staple pusher and approximation assemblies 50, 70 and the outer tube 340 is configured for selective reception over distal portions of staple pusher and approximation assemblies 50, 70.

With reference initially to FIG. 12, handle assembly 310 includes first and second handle sections 312a, 312b and a base member or hinge mechanism 330. First and second handle sections 312a, 312b are each pivotally connected to base member 330 at a proximal end 314a, 314b, respectively, thereof. In this manner, first and second handle sections 312a, 312b may be connected to base member 330 using pins (not shown), one or more hinges (not shown) or an other suitable means that permits pivoting of first and second handle sections 312a, 312b relative to base member 330. Pivotal separation of first and second handle sections 312a, 312b from each other facilitates removal of pusher assembly 50 and approximation assembly 70 from handle assembly 310 to permit sterilization of these components. Alternatively, first and second handle sections 312a, 312b may be integrally formed with base member 330. As such, first and second handle sections 312a, 312b may be connected to base member 330 by a living hinge.

With reference still to FIG. 12, distal ends 320a, 320b of first and second handle sections 312a, 312b, together define a threaded collar 320 which is configured for releasable engagement with a ferrule 345 when first and second handle sections 312a, 312b are in the closed configuration (FIG. 15). Ferrule 345 is slidably positioned about outer tube 340 to facilitate releasable engagement of the proximal end 340a of outer tube 340 with the distal end of handle assembly 310.

Base member 330 is configured to be received about proximal end 72a of rotatable sleeve 72 of approximation assembly 70. As discussed above, base member 330 is configured to pivotally engage first and second handle sections 312a, 312a. As shown, approximation knob 350 is securely attached to base member 330 in a rotatable manner. It is envisioned that approximation knob 350 may be releasably attached to base member 330 to facilitate separation and sterilization and/or to permit replacement of approximation knob 350 in the event of wear or damage.

With continued reference to FIG. 12, proximal end 340a of outer tube 340 includes a pair of openings 341 which are configured to receive respective protrusions 313a, 313b formed on an inner surface of distal end 320a, 320b of first and second handle sections 312a, 312b, respectively. As first and second handle sections 312a, 312b are moved from an open configuration (FIG. 14) to a closed configuration (FIG. 16), the protrusions are received within openings 341 to secure outer tube 340 to handle assembly 310. Ferrule 345 is configured to be received about proximal end 340a of outer tube 340 and releasably engage distal end 320 of handle assembly 310 to maintain handle assembly 310 in the closed configuration, thereby securing outer tube 340 to handle assembly 310.

With reference now to FIGS. 13-16, the attachment of housing assembly 300 to staple pusher assembly 50 and approximation assembly 70 will be described. Referring initially to FIG. 13, outer tube 340 and ferrule 345 are received over a distal end of staple pusher assembly 50 and approximation assembly 70. Turning to FIG. 14, handle assembly 310 is next attached to a proximal portion of approximation assembly 70 by receiving base member 330 about proximal end 72a (FIG. 13) of rotatable sleeve 72. As shown, approximation knob 350 is secured to base member 330, therefore, attachment of approximation knob 350 to proximal end 72a of rotatable sleeve 72 occurs concurrently with reception of proximal end 72a of rotatable sleeve 72 through base member 330.

With reference now to FIG. 15, first and second handle sections 312a, 312b of handle assembly 310 are then pivoted to the closed position, thereby encasing the proximal portions of staple pusher and approximation assemblies 50, 70. Prior to pivoting handle sections 312a, 312b to the closed configuration, proximal end 340a of outer tube 340 is positioned between distal ends 320a, 320b of first and second handle sections 312a, 312b, respectively. In this manner, outer tube 340 is secured to handle assembly 310. Once handle assembly 310 is in the closed configuration, ferrule 345 is affixed to distal end 320 of handle assembly 310 to maintain first and second handle sections 312a, 312b in the closed configuration (FIG. 16).

After use of the surgical stapling device, the components of the surgical stapling device can be disassembled for sterilization by reversing the steps outlined above. Briefly, ferrule 345 is disengaged from distal end 320 of handle assembly 310 and handle assembly 310 is moved to the open configuration (FIG. 14) by separating proximal ends 320a, 320b of first and second handle sections 312a, 312b. Once handle assembly 310 is in the open configuration, handle assembly 310 is disengaged from proximal end 72a of rotatable sleeve 72 of approximation assembly 70 by sliding base member 330 proximally relative thereto, as indicated by arrow “C”. Outer tube 340 and ferrule 345 may be removed from the distal ends of pusher staple and approximation assemblies 50, 70 at any time following the separation of first and second handle sections 312a, 312b. The components of each of housing assembly 300, staple pusher assembly 50 and approximation assembly 70 may then be sterilized in a traditional manner.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto. For example, although the description refers exclusively to staples, it is envisioned that staples may include different types of tissue fasteners including two-part fasteners. In a stapling device for applying two-part fastener, the anvil assembly of the stapling device would support one part of each two-part fastener.

1. A surgical stapling device comprising:
a staple pusher assembly;
an approximation assembly;
a handle assembly including first and second handle sections pivotally secured in relation to each other about a proximal portion of the staple pusher and approximation assemblies, the first and second handle sections being pivotal between an open configuration and a closed configuration, wherein in the open configuration, the first and second handle sections are pivoted to expose the proximal portion of the staple pusher and approximation assemblies;
an outer tube received about a distal portion of the staple pusher and approximation assemblies; and
a ferrule configured to be received about a proximal end of the outer tube and releasably connected to a distal end of the first and second handle sections to secure the outer tube to the handle assembly and retain the first and second handle sections in the closed configuration.

2. The surgical stapling device of claim 1, further including a knob releasably attached to a distal end of the approximation assembly.

3. The surgical stapling device of claim 3, further including a base, wherein the first and second handle sections are pivotally secured to the base.

4. The surgical stapling device of claim 3, wherein a longitudinal edge of each of the first and second handle sections is configured for pivotal engagement with the base.

5. The surgical stapling device of claim 3, wherein a proximal end of each of the first and second handle sections is configured for pivotal engagement with the base.

6. The surgical stapling device of claim 3, wherein a proximal extension of the base includes a pair of semi-circular clips configured to be releasably received about a rotatable sleeve of the approximation assembly.

7. The surgical stapling device of claim 3, wherein a distal extension of the base includes a semi-circular flange configured to be releasably received about an elongated pusher tube of the staple pusher assembly.

8. The surgical stapling device of claim 3, wherein the base includes a pair of support members positioned to stabilize a firing trigger of the staple pusher assembly.

9. The surgical stapling device of claim 1, wherein the ferrule is releasably connected to the distal end of the first and second handle sections by friction-fit.

10. The surgical stapling device of claim 1, wherein the ferrule is releasably connected to the distal end of the first and second handle sections by bayonet coupling.

11. The surgical stapling device of claim 1, wherein the ferrule is releasably connected to the distal end of the first and second handle sections by threads.

12. A method of assembling a surgical stapler comprising the steps of:
providing a staple pusher assembly, an approximation assembly, a housing assembly, an outer tube and a ferrule, wherein the housing assembly includes first and second handle sections;
receiving the outer tube and the ferrule over a distal end of the staple pusher and a distal end of the approximation assembly;
attaching the handle assembly to a proximal portion of approximation assembly;
positioning a proximal end of the outer tube between distal end of the first and second handle sections;
pivoting first and second handle sections of the handle assembly relative to each other to a closed position to encase the proximal portions of staple pusher and approximation assemblies; and
securing the ferrule to the distal ends of the first and second handle sections.

13. A method of disassembling a surgical stapler comprising the steps of:
providing a surgical stapler having a staple pusher assembly, an approximation assembly, a housing assembly, an outer tube and a ferrule, wherein the housing assembly includes first and second handle sections;
disengaging the ferrule from about distal ends of the first and second handle sections;
pivoting at least one of the first and second handle sections relative to each other to expose proximal ends of the staple pusher assembly and the approximation assembly;
removing the outer tube and ferrule from about distal ends of the staple pusher assembly and the approximation assembly; and
disengaging the housing assembly from the proximal ends of the staple pusher assembly and the approximation assembly.
